(19) United States
(12) Patent Application Publication
(10) Pub. No.: US 2025/0367126 A1
(43) Pub. Date: Dec. 4, 2025

(54) SOFTGEL CAPSULE COMPRISING A H2 RECEPTOR ANTAGONIST

(71) Applicant: KENVUE BRANDS LLC, Summit, NJ (US)

(72) Inventors: Bruce P. Smith, Blue Bell, PA (US); Linus G. Fonkwe, Waltham, MA (US)

(21) Appl. No.: 19/227,824

(22) Filed: Jun. 4, 2025

Related U.S. Application Data
(60) Provisional application No. 63/655,722, filed on Jun. 4, 2024.

(51) Int. Cl. A61K 9/48; A61K 9/00; A61K 9/10; A61K 31/426; A61K 47/14; A61K 47/44

(52) U.S. Cl. CPC A61K 9/4858; A61K 9/0053; A61K 9/10; A61K 9/4825; A61K 9/4875; A61K 31/426; A61K 47/14; A61K 47/44

(57) ABSTRACT

The disclosure generally relates to soft gelatin oral dosage form. In particular the disclosure relates to an oral dosage form comprising a soft gelatin capsule and a liquid fill, where the liquid fill is a suspension of an H2 receptor antagonist in a hydrophobic/lipophilic excipient. The disclosure also relates to a method of treating a gastric disease or disorder by using said oral dosage form.

### PRIORITY CLAIM

The present application claims priority to U.S. Provisional Application Ser. No. 63/655,722 filed Jun. 4, 2024, the entire contents of which is hereby incorporated by reference herein.

### FIELD

The disclosure generally relates to soft gelatin oral dosage forms. In particular, the disclosure relates to an oral dosage form having a soft gelatin capsule and a liquid fill, where the liquid fill is a suspension of an H2 receptor antagonist in a hydrophobic/lipophilic excipient. The disclosure also relates to a method of treating a gastric disease or disorder by use of said oral dosage form.

### BACKGROUND

Histamine H2-receptor antagonists, for example cimetidine, ranitidine, nizetidine, roxatine and famotidine, reduce acid secretion by acting directly on the acid-secreting parietal cell located within the gastric gland of the stomach wall. Histamine H2-receptor antagonists can be formulated in an oral dosage form which may disintegrate/disperse in the buccal cavity, for example chewable tablets or fast dissolving tablets; or in a swallowable dosage form such as a hard tablet, capsule or the like.

Histamine H2-receptor antagonists, such as famotidine, are bitter in taste. When incorporated into oral dosage forms, which disintegrate/disperse in the buccal cavity, Histamine H2-receptor antagonists such as famotidine require effective taste-masking to be more appealing to the user.

Swallowable dosage forms are another option, they will release the Histamine H2-receptor antagonists directly in the stomach and thus don't require taste masking.

Capsules are solid dosage forms in which the drug substance and/or excipients are enclosed within a soluble container or shell, or are coated on the capsule shell. The shells may be composed of two pieces: a body and a cap, or they may be composed of a single piece. Two-piece capsules are commonly referred to as hard-shell capsules, and one-piece capsules are often referred to as soft-shell capsules. This two-piece and one-piece capsule distinction, although imprecise, reflects differing levels of plasticizers in the two compositions and the fact that one-piece capsules typically are more pliable than two-piece capsules. The shells of capsules are usually made from gelatin. However, they also may be made from cellulose polymers (e.g., hypromellose) or other suitable materials. Two-piece capsules may include two telescoping cap and body pieces in a range of standard sizes. One-piece capsules typically are used to deliver a drug substance as a solution or suspension. Liquid formulations placed into one-piece capsules may offer advantages by comparison with dry-filled capsules and tablets in achieving content uniformity of potent drug substance(s) or acceptable dissolution of drug substance(s) with poor aqueous solubility. The most common type of one-piece capsule is that produced by a rotary die process that results in a capsule with a seam. The soft gelatin shell of a one-piece capsule is somewhat thicker than that of two-piece capsules and is plasticized by the addition of polyols such as glycerin, sorbitol, or other suitable materials.

Hard tablets or hard-shell capsules, due to their texture and hardness, may be unappropriated or unpleasant for some consumer that have difficulties to swallow.

Soft-shell capsules, or soft gelatin capsules, are more flexible (pliable) and can provide a more pleasant experience to the users that prefer swallowable dosage forms. Soft gelatin capsules can be even more flexible when filled with a liquid. Liquid filled gelatin capsules may also provide a faster release of the active ingredient present in the liquid fill once the gelatin capsule is dissolved in the stomach.

Finally, the formulation of Histamine H2-receptor antagonists such as famotidine in a liquid media is often difficult because of famotidine poor lipophilicity, poor aqueous solubility, and low stability outside a pH range of 6 to 7 (J Pharm Pharmacol 1993, 45:682-686).

The present disclosure addresses the above-described unmet needs by providing a liquid Histamine H2-receptor antagonist formulation in a soft gelatin capsule, where the Histamine H2-receptor antagonist is stable over a longer period of time, the capsule contains a higher amount of Histamine H2-receptor, the capsule is easier to swallow, and/or the capsule is perceived to have a faster action on symptom relief, as compared to other dosage forms.

### SUMMARY

One aspect of the disclosure pertains to an oral dosage form comprising a soft gelatin capsule and a liquid fill contained within the soft gelatin capsule, where the liquid fill comprises a suspension of an H2 receptor antagonist in a hydrophobic/lipophilic excipient substantially free from water, and where at least 50% in wt % of the hydrophobic/lipophilic excipient consists of linoleic acid or linoleate moiety.

This aspect may be combined with a variety of embodiments, in any combination. Thus, in some embodiments the hydrophobic/lipophilic excipient has a HLB value below 3, preferably below 2, preferably at a maximum of 1. In some embodiments the hydrophobic/lipophilic excipient consists of glyceryl monolinoleate, corn oil, or combinations thereof. In some embodiments, a solubility of the H2 receptor antagonist in the hydrophobic/lipophilic excipient is below 1 mg/g. In some embodiments, the H2 receptor antagonist is selected from the group consisting of cimetidine, ranitidine, nizatidine, roxatidine and famotidine, and pharmaceutically acceptable salts thereof. In some embodiments, the H2 receptor antagonist comprises famotidine. In some embodiments, the H2 receptor antagonist comprises at least 15%, in wt %, of the liquid fill. In some embodiments, the H2 receptor antagonist comprises at least 5 mg per dosage form, preferably at least 10 mg. In some embodiments, after 24 months at least 95% of the H2 receptor antagonist has not degraded.

Another aspect of the disclosure pertains to an oral dosage form, comprising a soft gelatin capsule and a liquid fill contained within the soft gelatin capsule, where the liquid fill consists of a suspension of famotidine in a hydrophobic/lipophilic excipient substantially free from water, and where the hydrophobic/lipophilic excipient consists of glyceryl monolinoleate, corn oil or combinations thereof.

Another aspect of the disclosure pertains to an oral dosage form according to any of the embodiments described above, comprising a soft gelatin capsule and a liquid fill contained within the soft gelatin capsule, where the liquid fill consists of a suspension of famotidine in a hydrophobic/lipophilic excipient substantially free from water, and where the hydrophobic/lipophilic excipient consists of glyceryl monolinoleate.

Another aspect of the disclosure pertains to an oral dosage form according to any of the embodiments described above, comprising a soft gelatin capsule and a liquid fill contained within the soft gelatin capsule, where the liquid fill consists of a suspension of famotidine in a hydrophobic/lipophilic excipient substantially free from water, and where the hydrophobic/lipophilic excipient consists of corn oil.

Another aspect of the disclosure pertains to a method of treating a gastric disease or disorder by use of the oral dosage form according to any of the embodiments described above.

### DETAILED DESCRIPTION

As used herein, a “soft gelatin capsule” is a single-unit solid dosage form, soft capsules made from gelatin, consisting of a liquid or semi-solid fill enveloped by a one-piece hermetically sealed elastic outer shell. Soft gelatin capsules are formed, filled in one continuous operation, preferably by the rotary die process. Soft gelatin capsules, or “softgels,” are water sensitive and dissolve when placed in an aqueous media or in a formulation containing more than 20% water; they are also pH sensitive as the gelatin will be hydrolyzed at low pH (2-3).

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The general convention in the scientific and technical literature is applied: the last decimal place of a numerical value indicates its degree of accuracy. Where no other error margins are given, the maximum margin is ascertained by applying the rounding-off convention to the last decimal place, for example for a measurement of 3.5%, the error margin is 3.45-3.54.

All percentages (%) are by weight (wt %) unless otherwise specified herein.

One aspect of the disclosure pertains to an oral dosage form comprising a soft gelatin capsule and a liquid fill contained within the soft gelatin capsule, where the liquid fill comprises a suspension of an H2 receptor antagonist in a hydrophobic/lipophilic excipient substantially free from water, and where at least 50%, in wt %, of the hydrophobic/lipophilic excipient consists of linoleic acid or linoleate moiety.

Surprisingly, it has been discovered that a H2 receptor antagonist, is stable over a long period of time, at least 1 year, or even up to 2 years, when placed in a hydrophobic/lipophilic excipient that consists in majority, at least 50%, of linoleic acid or linoleate moiety.

By linoleic acid or linoleate moiety it is meant a linoleic acid, or an ester or salt of linoleate. When considering an ester of linoleate, at least 50% in weight of the said ester consists of at least one linoleate moiety. Linoleic acid corresponds to the formula: HOOC(CH₂)—CH═CHCH₂CH═CH(CH₂)₄CH₃ where the two alkene double bounds are in cis configuration. Linoleic acid is a fatty acid sometimes denoted 18:2 (n−6) or 18:2 cis-9,12. A linoleate is a salt or ester of this acid. By extension a linoleate moiety corresponds to the formula: OOC(CH₂)₇CH—CHCH₂CH—CH(CH₂)₄CH₃ where the two alkene double bounds are in cis configuration.

As used herein, “essentially free” or “substantially free” of water means containing less than 1%, or less than 0.1%, or less than 0.01%, or less than 0.001%, or the total absence of water in the liquid fill; where % are in weight percentage (wt %) of the liquid fill.

In another embodiment the oral dosage form may comprise a soft gelatin capsule and a liquid fill contained within the soft gelatin capsule, where the liquid fill consists of a suspension of an H2 receptor antagonist in a hydrophobic/lipophilic excipient substantially free from water, and where at least 50%, in wt %, of the hydrophobic/lipophilic excipient consists of linoleic acid or linoleate moiety.

In one or more embodiments, the hydrophobic/lipophilic excipient may have a HLB value below 3, preferably below 2, preferably at a maximum of 1.

Excipients with a low HLB value were found to be particularly beneficial in the scope of the present disclosure because of their low affinity for water, thus low content of water; and for their low solubilization of Histamine H2-receptor antagonists such as famotidine.

Low solubility of Histamine H2-receptor antagonists such as famotidine allows for the preparation of suspension and not solutions. Histamine H2-receptor antagonists such as famotidine are extremely sensitive to humidity and can degrade.

In one or more embodiments, the hydrophobic/lipophilic excipient may be selected among oils or natural oil having a linoleic fatty acid content of at least 50%, or esters of linoleate where the linoleate moiety accounts for at least 50% in weight of the excipient total weight.

In one or more embodiments the hydrophobic/lipophilic excipient may be selected among linoleate glyceryl esters or natural oils such as corn oil, cotton seed oil, grape seed oil, hemp seed oil, soy bean oil, walnut oil or combination thereof; preferably the hydrophobic/lipophilic excipient consists of glyceryl monolinoleate, corn oil or combinations thereof.

Any natural oil, acceptable for human consumption, may be used as hydrophobic/lipophilic excipient in the scope of the present disclosure provided its content in linoleic fatty acid is more or equal to 50%, in wt %.

Preferably the hydrophobic/lipophilic excipient may be glyceryl monolinoleate.

Preferably the hydrophobic/lipophilic excipient may be corn oil, such as super refined corn oil.

In one or more embodiments a solubility of the H2 receptor antagonist in the hydrophobic/lipophilic excipient may be below 1 mg/g, or preferably below 0.1 mg/g, or even more preferably below 0.01 mg/g.

In one or more embodiments the solubility of famotidine in the hydrophobic/lipophilic excipient may be below 1 mg/g, or preferably below 0.1 mg/g, or even more preferably below 0.01 mg/g.

For clarification, the mass of the H2 receptor antagonist, such as famotidine, soluble in the hydrophobic/lipophilic excipient may be less than 1 mg per gram (g) of said excipient, preferably less than 0.1 mg per g of said excipient, even more preferably less than 0.01 mg per g of said excipient.

Surprisingly, it has been observed by the inventors that H2 receptor antagonists such as famotidine are less susceptible of degradation (i.e. are more stable) when formulated in a suspension compared to a solution.

As used herein, a “suspension” means a heterogenous mixture of a liquid (for example said hydrophobic/lipophilic excipient) containing solid particles (for example said H2 receptor antagonist such as famotidine).

In one or more embodiments the H2 receptor antagonist may be selected from the group consisting of cimetidine, ranitidine, nizatidine, roxatidine and famotidine, and pharmaceutically acceptable salts thereof.

In one or more embodiments the H2 receptor antagonist may comprise famotidine.

In one or more embodiments the H2 receptor antagonist may consists of famotidine.

In one or more embodiments the suspension of H2 receptor antagonist may consist of H2 receptor antagonist particles having a diameter ranging from 1 μm to 50 μm. In one or more embodiments the suspension of H2 receptor antagonist may consist of famotidine particles having a diameter ranging from 1 μm to 50 μm.

In one or more embodiments the H2 receptor antagonist may comprise at least 15%, in wt %, of the liquid fill. In other words, at least 15%, in wt %, of the liquid fill may consist of the H2 receptor antagonist.

For example, the H2 receptor antagonist may comprise at least 16%, or at least 17%, or at least 18% in wt % of the liquid fill.

In certain embodiments famotidine may be the H2 receptor antagonist, and may comprise at least 15%, or at least 16%, or at least 17%, or at least 18%, in wt % of the liquid fill.

It is desirable to have a liquid fill with the highest possible content of H2 receptor antagonist, such as famotidine, as for a given amount of H2 receptor antagonist it would enable to prepare a smaller oral dosage form thus easier to swallow.

In one or more embodiments the H2 receptor antagonist may comprise at least 5 mg per oral dosage form, preferably at least 10 mg. In other words, at least 5 mg per oral dosage form may consist of the H2 receptor antagonist.

In certain embodiments famotidine may comprise at least 5 mg per oral dosage form, preferably at least 10 mg.

In one or more embodiments H2 receptor antagonist may comprise 40 mg or less, per oral dosage form. Preferably, H2 receptor antagonist may comprise from 10 mg to 40 mg, or from 10 mg to 20 mg, or from 20 mg to 40 mg per oral dosage form.

In one or more embodiments famotidine may comprise 40 mg or less, per oral dosage form. Preferably, famotidine may comprise from 10 mg to 40 mg, or from 10 mg to 20 mg, or from 20 mg to 40 mg per oral dosage form.

In one or more embodiments, after 24 months, at least 95% of the H2 receptor antagonist may not have degraded.

In other words, the stability of H2 receptor antagonist after 24 months may be more than 95%. This percentage is relative to the amount of H2 receptor antagonist used when the liquid fill was formulated.

For example, the total amount of impurities from degradation of the H2 receptor agonist, in the liquid fill of a soft gelatin capsule as disclosed in the present disclosure, analyzed by HPLC, may be less than 5% after a period of 24 months at 25° C. and 60% relative humidity.

In certain embodiment the stability of famotidine after 24 months may be more than 95%.

For example, the total amount of impurities from degradation of famotidine, in the liquid fill of a soft gelatin capsule as disclosed in the present disclosure, analyzed by HPLC, may be less than 5% after a period of 24 months at 25° C. and 60% relative humidity.

It is highly desirable to have a H2 receptor antagonist, such as famotidine, be as stable as possible over time as it will improve the shelf life of the oral dosage form, reducing waste and offering the user a quality product even after long conservation. This is particularly desirable for unstable products, such as those containing famotidine, which are extremely sensitive to humidity and can degrade easily.

In a preferred embodiment the oral dosage form according to the present disclosure may comprise a soft gelatin capsule and a liquid fill contained within the soft gelatin capsule, where the liquid fill consists of a suspension of famotidine in a hydrophobic/lipophilic excipient substantially free from water, and where the hydrophobic/lipophilic excipient consists of glyceryl monolinoleate, corn oil or combinations thereof.

In a preferred embodiment the oral dosage form according to the present disclosure may comprise a soft gelatin capsule and a liquid fill contained within the soft gelatin capsule, where the liquid fill consists of a suspension of famotidine in a hydrophobic/lipophilic excipient substantially free from water, and where the hydrophobic/lipophilic excipient consists of glyceryl monolinoleate.

In a preferred embodiment the oral dosage form according to the present disclosure may comprise a soft gelatin capsule and a liquid fill contained within the soft gelatin capsule, where the liquid fill consists of a suspension of famotidine in a hydrophobic/lipophilic excipient substantially free from water, and where the hydrophobic/lipophilic excipient consists of corn oil.

Another aspect of the disclosure pertains to a method of treating a gastric disease or disorder by use of the oral dosage form according to any of the embodiments disclosed herein.

As used herein the term “gastric disease or disorder” may include, but is not limited to, heartburn, gastric and duodenal ulcers, gastrointestinal hypersecretory conditions, gastroesophageal reflux disease and/or esophagitis.

In certain embodiments the oral dosage form according to any of the embodiments disclosed in the present disclosure, may be suitable for use in the treatment of gastric disease or disorder.

The disclosure may pertain to the use of the oral dosage form according to any of the embodiments disclosed above for the manufacture of a medicament for the treatment of gastric disease or disorder.

The following Examples are intended to illustrate, but not to limit, the disclosed subject matter in any manner, shape, or form, either explicitly or implicitly.

### Examples

### 1—Selection of Solvent

A famotidine solubility screen was performed using various liquid excipients commonly used in formulating fill materials for liquid-filled encapsulation. A list of excipients used for exploring the solubility of famotidine is shown in Table 1. For this evaluation, excipients were added to clear glass vials at ambient temperature. Enough famotidine was added to the liquid excipients to create saturated solutions/dispersions. The vials were attached to a Burrell Action Wrist Shaker (mechanical shaker) and agitated for approximately 60 hours (hrs) at ambient conditions. During the shaking period, extra famotidine was added to the vials as needed to maintain excess famotidine (saturated) conditions in the vials.

After shaking for approximately 60 hrs, a first portion of the supernatant solution from each vial was submitted for chemical analysis. A second portion of the remaining dispersions was transferred to amber vials and stored at ambient conditions. Both clear vials (containing saturated solutions with excess famotidine) and amber vials (containing saturated famotidine solutions/dispersions with no excess famotidine) were stored for 2 weeks at ambient conditions. Solutions from both amber and clear vials were centrifuged and analyzed for solubilized famotidine content using an analytical method described in the paragraph “Chemical stability” below.

TABLE 1
Excipients for solubility screen

| Excipient/Solvent Vehicle for Solubility Screen | Supplier | HLB |
|---|---|---|
| Polyoxyl-35 Castor Oil NF (Kolliphor ® EL) | BASF Corporation | 12-14 |
| Polysorbate 80 NF (Tween 80) | Croda International oda | 15 |
| Polyethylene Glycol 400 NF (PEG 400) | DOW Chemical | N/A |
| Medium Chain Triglycerides NF (MCT) | Abitec Corporation | 1 |
| Caprylocaproyl Polyoxyl-8 Glycerides NF | Gattefosse Corporation | 12 |
| (Labrasol ®) | | |
| Oleic acid | Croda International | 1 |
| Mono/Diglycerides of Capric/Caprylic Acid NF | Abitec Corporation | 6-7 |
| (Capmul MCM) | | |
| Corn Oil, super refined NF | Gattefosse Corporation | 1 |
| Glacial acetic acid | | N/A |
| Acidified PEG 400 solution with water | DOW Chemical | N/A |
| [PEG 400 (59.7%) + acetic acid (30.0%) + water | | |
| (10.3%)] | | |
| Glyceryl monolinoleate NF (Maisine CC) | Gattefosse Corporation | 1 |
| Propylene glycol USP | | N/A |

Equilibrium solubility data for famotidine after 60 hrs of shaking (initial) and after 2 weeks of storage in clear and amber vials at ambient conditions are shown in Table 2. High solubility was found in hydrophilic solvents (PEG 400, acidified PEG 400 solution with water, glacial acetic acid). Acceptable solubility values were found in high HLB (hydrophilic) surfactants (kolliphor EL and polysorbate 80), labrasol and oleic acid (hydrophobic solvent). No significant amount of solubilization was found in hydrophobic solvents with low HLB values-corn oil, capmul MCM and maisine CC.

Storing saturated solutions of famotidine in clear and amber vials did not change equilibrium solubility for most solvents. The increase in famotidine content in oleic acid after 2 weeks of storage in clear vials was likely due to continued solubilization of famotidine in oleic acid after the original 60 hrs of shaking. There was no difference in famotidine content in solutions due to storage in clear versus amber vials, indicating that the famotidine solutions may not have undergone degradation due to ambient light during the duration of this study. Decrease in solubilized famotidine content in acidified PEG 400 solution with water was likely due to hydrolysis of famotidine in the presence of water. Analysis of vials with famotidine in glacial acetic acid and propylene glycol was discontinued after initial testing because these solvents may not be suitable for manufacturing softgel dosage form when used in high concentrations.

TABLE 2
Equilibrium solubility of famotidine in various liquid

| | | | mg/g | mg/g | mg/g | mg/g |
|---|---|---|---|---|---|---|
| Excipient/Solvent | mg/g | mg/g | Total | Solvent | Total | Solvent |
| Vehicles Excipient/Solvent | Total | Solvent | 2 Weeks in | | 2 Weeks in | |
| Vehicle | Initial | | Clear Vials | | Amber Vials | |
| Polyoxyl-35 Castor Oil NF | 60 | 64 | 60 | 63 | 60 | 64 |
| (Kolliphor EL) | | | | | | |
| Polysorbate 80 NF | 43 | 45 | 35 | 36 | 43 | 45 |
| (Tween 80) | | | | | | |
| Medium Chain Triglycerides | ND | ND | ND | ND | ND | ND |
| NF | | | | | | |
| (MCT) | | | | | | |
| Caprylocaproyl Polyoxyl-8 | 57 | 61 | 57 | 61 | 55 | 58 |
| Glycerides NF | | | | | | |
| (Labrasol) | | | | | | |
| Oleic acid | 52 | 55 | 72 | 78 | 53 | 56 |
| HLB = 1 | | | | | | |
| Mono/Diglycerides of | 3 | 3 | 3 | 3 | 3 | 3 |
| Capric/Caprylic Acid NF | | | | | | |
| (Capmul MCM) | | | | | | |
| Corn oil, super refined NF | ND | ND | ND | ND | ND | ND |
| HLB = 1 | | | | | | |
| Glyceryl monolinoleate NF | ND | ND | ND | ND | ND | ND |
| (Maisine CC) | | | | | | |
| HLB = 1 | | | | | | |
| Polyethylene Glycol 400, NF | 106 | 118 | 104 | 116 | 103 | 115 |
| (PEG 400) | | | | | | |
| Acidified PEG 400 Solution | 138 | 160 | 124 | 141 | 121 | 137 |
| with water | | | | | | |
| [PEG 400 (59.7%) + acetic acid | | | | | | |
| (30.0%) + water (10.3%)] | | | | | | |
| Glacial acetic acid | 241 | 318 | — | — | — | — |
| Propylene Glycol USP | 17 | 17 | — | — | — | — |

“ND” means not detected, famotidine content was below 0.1 mg/g.
“mg/g Total” refers to the calculation based on the binary mixture (solvent and famotidine)
“mg/g Solvent” refers to the calculation based on the solvent used in the binary mixture.

### 2—Formulation of Solution/Suspension Fill

Based on excipient solubility data in Table 2, both solution-type and suspension-type fill materials were planned for evaluation. Excipients with high famotidine solubility were chosen for solution-type formulations while for suspension-type formulations, excipients with lowest famotidine solubility were chosen.

Hydrophilic PEG 400 and highly hydrophobic oleic acid were chosen as the main solvents for solution-type formulations

Corn oil and maisine CC (highly hydrophobic excipients) were chosen as vehicles for suspension-type formulations.

### 2.1—Solution Fills

For solution-type formulations, excipients with the highest famotidine solubility were chosen. The highly hydrophilic PEG 400 and highly hydrophobic oleic acid were chosen as base solvents for famotidine solutions. Since the solubility data in Table 2 are equilibrium solubility values under saturated conditions, they are not optimal for developing solution-type fill formulations. This is because such solutions would be unstable and prone to famotidine precipitation during storage. Thus, the amount of solvent to solubilize 20 mg famotidine in Table 2 was calculated and increased by more than 15%. After adjusting the amounts of excipients to minimize potential for famotidine precipitation in PEG 400 and oleic acid solutions, the two solution-type fill formulations in Table 3 were prepared.

TABLE 3
Compositions of famotidine solution fill formulations

| | Solution Fill | | | |
|---|---|---|---|---|
| | Example 1 (comparative) | | Example 2 (comparative) | |
| Ingredient | mg/capsule | % | mg/capsule | % |
| Famotidine | 20.00 | 8.01 | 20.00 | 4.08 |
| PEG 400 | 229.81 | 91.99 | — | — |
| Oleic acid | — | — | 470.23 | 95.92 |
| Total | 249.81 | 100.00 | 490.23 | 100.00 |

### 2.2—Suspension Fills

For suspension-type formulations, excipients with lowest famotidine solubility were chosen. The highly hydrophobic excipients maisine CC and corn oil, with no detectable amount of solubilized famotidine, were chosen as vehicles for famotidine suspension.

Glyceryl distearate was used as a thickener and lecithin was used as a dispersant to the base formulations. A blend of solvent, a combination of glyceryl distearate and lecithin, was prepared. famotidine was slowly added to the mix and flowability of the mix was evaluated visually at ambient temperature. Final suspension formulations, judged to be initially suitable from preliminary evaluation, are shown in Table 4.

Both formulations were identical except for the base lipids used (maisine CC and corn oil). Both formulations had similar appearance.

TABLE 4
Compositions of famotidine suspension fill formulations

| | Suspension Fill | | | |
|---|---|---|---|---|
| | Example 3 (inventive) | | Example 4 (inventive) | |
| Ingredient | mg/capsule | % | mg/capsule | % |
| Famotidine | 20.00 | 18.19 | 20.00 | 18.19 |
| Maisine CC | 87.02 | 79.15 | — | — |
| Corn oil | — | — | 87.04 | 79.16 |
| Precirol ATO | 1.42 | 1.29 | 1.42 | 1.29 |
| (glyceryl distearate) | | | | |
| Lecithin | 1.51 | 1.37 | 1.50 | 1.36 |
| Total | 109.94 | 100.00 | 109.960 | 100.00 |

### 3—Chemical Stability

Stability of famotidine solutions (Examples 1 and 2) and suspensions (Examples 3 and 4) were evaluated.

Famotidine solutions (Examples 1 and 2) and suspensions (Examples 3 and 4) were stored at ambient and at 60° C. temperatures. All samples were analyzed after 2 and 4 weeks of storage.

Method: The famotidine assays were analyzed versus an assay working standard famotidine solution prepared at 80 μg/mL and famotidine impurity assays were analyzed versus a degradation products standard solution prepared at 1.0% FAM (0.8 μg/mL), using an HPLC.

HPLC analysis was implemented on an Advanced Chromatography Technologies (ACE) Column C8, 3 μm (150 mm×4.6 mm I.D.), ACE-112-1546 or EXL-112-1546U. The mobile phase was a gradient of 40 mM KPF6 in 10 mM sodium phosphate buffer versus acetonitrile; the gradient was from 98:2 to 30:70, where the ratio are expressed in volume, and the gradient was implemented over 26 minutes. The flow rate was 1.0 mL/min, and the injection volume 15 μL. The UV detector was set at 278 nm. The column temperature was 35° C. The run time was about 26 min.

Multiple degradants were examined on stability.

### 3.1—Famotidine Solution in PEG 400

Assay and impurities data for famotidine solutions in PEG 400 during storage are presented in Table 5.

As shown in Table 5, there was a gradual decrease in assay and a corresponding gradual increase in impurities during storage at ambient conditions. This indicates that there may have been some hydrolysis of famotidine in solution during storage at ambient conditions. There was significant decrease in assay with significant increase in impurities (total and known impurities) during storage of famotidine solution at 60° C., as shown in Table 5.

TABLE 5
Analysis of famotidine solution fill formulations in PEG 400

| | Time Point | | | | | |
|---|---|---|---|---|---|---|
| | | 2 weeks | 4 weeks | | 2 Weeks | 4 weeks |
| | | at | at | | at | at |
| | Initial | ambient | ambient | Initial | 60° C. | 60° C. |
| Assay (mg/cap) | 19.0 | 19.0 | 18.8 | 19.0 | 17.3 | 16.2 |
| Assay (%) | 95.2 | 94.8 | 93.9 | 95.2 | 86.4 | 80.8 |
| Total | 4.41 | 5.62 | 6.00 | 4.41 | 11.51 | 17.46 |
| Impurities¹ (%) | | | | | | |
| FAM-A1² | 0.16 | 0.17 | 0.17 | 0.16 | 0.07 | 0.13 |
| FAM-A2² | NA | NA | NA | NA | NA | NA |
| FAM-A3² | 0.18 | 0.17 | 0.17 | 0.18 | 0.22 | 0.38 |
| FAM-A6² | 1.17 | 1.12 | 1.10 | 1.17 | 0.10 | 0.08 |

¹Total Impurities include all peaks that were detected
²Known impurities of famotidine

### 3.2—Famotidine Solution in Oleic Acid

Assay and impurities data for famotidine solutions in oleic acid are presented in Table 6.

As shown in Table 6, initial assay values were low indicating that there may have been some degradation of famotidine in oleic acid between fill preparation and analysis. There was gradual decrease in assay values and increase in impurities during storage at ambient conditions with the amount of total impurities being very high. This indicates that there was hydrolysis of famotidine in oleic acid during storage at ambient conditions. There was significant decrease in assay with significant increase in impurities during storage of famotidine solution at 60° C. The data indicates there was very little famotidine in oleic acid solution after about 2 weeks of storage at 60° C. This may be attributed to the fact that there was precipitation of famotidine observed in oleic acid solution during storage at 60° C. with no famotidine in solution.

TABLE 6
Analysis of famotidine solution in oleic acid

| | Time Point | | | | | |
|---|---|---|---|---|---|---|
| | | 2 weeks | 4 weeks | | 2 Weeks | 4 week |
| | | at | at | | at | at |
| | Initial | ambient | ambient | Initial | 60° C. | 60° C. |
| Assay (mg/cap) | 16.4 | 16.3 | 15.5 | 16.4 | 6.7 | 3.4 |
| Assay (%) | 81.8 | 81.4 | 77.7 | 81.8 | 33.5 | 17.2 |
| Total | 2.09 | 3.16 | 5.21 | 2.09 | 19.44 | 41.85 |
| Impurities¹ (%) | | | | | | |
| FAM-A1² | 0.81 | 1.80 | 3.67 | 0.81 | 7.02 | 3.64 |
| FAM-A2² | NA | NA | NA | NA | 0.58 | 1.10 |
| FAM-A3² | 0.15 | 0.13 | 0.16 | 0.15 | 6.06 | 11.21 |
| FAM-A6² | 0.22 | 0.33 | 0.45 | 0.22 | 0.37 | 0.29 |

¹Total Impurities include all peaks that were detected
²Known impurities of famotidine

### 3.3—Famotidine Suspension in Glyceryl Monolinoleate (Maisine CC)

Assay and impurities data for famotidine solutions in glyceryl monolinoleate are presented in Table 7.

As shown in Table 7, there was no change in assay and impurities during storage of famotidine suspensions in maisine CC at both ambient and 60° C., assay values remained high while impurity levels (total impurities and known impurities) remained very low. This indicated minimal to no hydrolysis of famotidine during storage of these suspensions at ambient temperature and 60° C. based on the analysis using preliminary analytical methods.

TABLE 7
Analysis of famotidine suspension in
glyceryl monolinoleate (maisine CC)

| | Time Point | | | | | |
|---|---|---|---|---|---|---|
| | | 2 weeks | 4 weeks | | 2 Weeks | 4 weeks |
| | | at | at | | at | at |
| | Initial | ambient | ambient | Initial | 60° C. | 60° C. |
| Assay (mg/cap) | 19.3 | 19.2 | 19.5 | 19.3 | 19.5 | 19.5 |
| Assay (%) | 96.7 | 95.8 | 97.5 | 96.7 | 97.7 | 97.7 |
| Total | 0.62 | 0.76 | 0.97 | 0.62 | 0.82 | 0.94 |
| Impurities¹ (%) | | | | | | |
| FAM-A1² | 0.08 | 0.10 | 0.10 | 0.08 | 0.14 | 0.15 |
| FAM-A2² | NA | NA | NA | NA | NA | NA |
| FAM-A3² | 0.20 | 0.18 | 0.19 | 0.20 | 0.19 | 0.19 |
| FAM-A6² | 0.27 | 0.42 | 0.58 | 0.27 | 0.23 | 0.15 |

¹Total Impurities include all peaks that were detected
²Known impurities of famotidine

### 3.4—Famotidine Suspension in Corn Oil

Assay and impurities data for famotidine solutions in corn oil are presented in Table 8.

As shown in Table 8, there was no change in assay and impurities during storage of famotidine suspensions in corn oil at both ambient and 60° C., assay values remained high while impurity levels (total impurities and known impurities) remained very low. This indicated minimal to no hydrolysis of famotidine during storage of these suspensions at ambient temperature and 60° C. based on the analysis using preliminary analytical methods.

TABLE 8
Analysis of famotidine suspension in corn oil.

| | Time Point | | | | | |
|---|---|---|---|---|---|---|
| | | 2 weeks | 4 weeks | | 2 Weeks | 4 week |
| | | at | at | | at | at |
| | Initial | ambient | ambient | Initial | 60° C. | 60° C. |
| Assay (mg/cap) | 19.4 | 19.1 | 19.1 | 19.4 | 19.6 | 19.5 |
| Assay (%) | 97 | 95.6 | 95.5 | 97 | 98.2 | 97.7 |
| Total | 0.39 | 0.42 | 0.46 | 0.39 | 0.55 | 0.84 |
| Impurities¹ (%) | | | | | | |
| FAM-A1² | 0.08 | 0.10 | 0.09 | 0.08 | 0.11 | 0.11 |
| FAM-A2² | 0.00 | 0.00 | 0.00 | 0.00 | 0.00 | 0.00 |
| FAM-A3² | 0.19 | 0.18 | 0.20 | 0.19 | 0.19 | 0.21 |
| FAM-A6² | 0.05 | 0.08 | 0.11 | 0.05 | 0.19 | 0.29 |

¹Total Impurities include all peaks that were detected
²Known impurities of famotidine

### 3.5—Conclusion for Stability

The solubility of famotidine was evaluated in various solvents. Based on solubility data, excipients were chosen for solution and suspension-type fill formulations. Famotidine solutions in PEG 400 and oleic acid were found to be chemically unstable as the famotidine was reported to potentially hydrolyze during storage, especially at 60° C. Famotidine solution in oleic acid appeared to be more susceptible to degradation. Famotidine suspensions, on the other hand, were found to be potentially more chemically stable during storage at ambient conditions and 60° C.

### 4—Famotidine Softgel Batches/Manufacture & Other Properties

### 4.1—Manufacture

### 4.1.1—Famotidine Fill Manufacture

Two famotidine, 20 mg fill formulations were used to manufacture famotidine softgels. A nominal yield of approximately 3,000 softgels (450 g of fill material) was achieved. A common white gel mass was used for all encapsulations. Batch quantities and ingredient information are shown in Table 9.

TABLE 9
Batch famotidine fill formulations

| | | | | Example 6 | | Example 7 | |
|---|---|---|---|---|---|---|---|
| | | | | (inventive) | | (inventive) | |
| | Theoretical batch quantity (g) | | | Qty | | Qty | |
| | mg/ | | g/ | Dispensed | | Dispensed | |
| Ingredient | capsule | % | Batch | g | | g | |
| Famotidine, USP | 20.000 | 13.33 | 60.000 | 60.0 | g | 60.0 | g |
| Corn oil, NF | 125.553 | 83.70 | 376.660 | 376.8 | g | | |
| Glyceryl monolinoleate, | | | | | | 376.7 | g |
| NF (Maisine CC) | | | | | | | |
| Glycerol distearate, NF | 2.280 | 1.52 | 6.840 | 6.7 | g | 6.8 | g |
| (Precirol ATO) | | | | | | | |
| Lecithin, NF | 2.167 | 1.44 | 6.500 | 6.6 | g | 6.5 | g |
| Total | 150.000 | 100.00 | 450.00 | 450.1 | g | 450.0 | g |

Famotidine fill formulations were manufactured according to the process below. As per fill composition corn oil, maisine CC, lecithin and precirol ATO 5 were dispensed into a clean stainless steel container with mixing. The dispersions were heated at 70-75° C. until precirol ATO 5 was completely molten, then cooled to not more than 40° C. with mixing. Famotidine was added and mixing was maintained for not less than 30 minutes to form a uniform, lump-free dispersion. The lump-free dispersion was de-aerated for not less than 30 minutes to form a suspension free of air bubbles. This suspension was used as fill material and stored at ambient temperature under a nitrogen blanket until encapsulation.

### 4.1.2—Gelatine Capsule Shell

Ingredients for the capsule shell were: gelatin (150 bloom lime bone gelatin, NF), glycerin, titanium dioxide (as colorant/opacifier) and purified water.

Manufacturing procedure of the capsule shell mass was as follows: Purified water and plasticizer were dispensed into a gel reactor. Gelatin was added to the gel reactor and mixed. Then, titanium dioxide mass was added and mixed. The resulting gel mass was heated at 65-80° C. for not less 30 minutes under vacuum until free of air bubbles. The de-aerated gel mass was stored overnight in stainless steel containers at 54 to 60° C. (130-140° F.) prior to encapsulation. The capsule shell mass was opaque and milky-white in color.

### 4.1.3—Manufacture of Famotidine 20 mg Softgels (Encapsulation)

Famotidine fill materials were encapsulated with capsule shell material (gel mass) using 3E oval dies with two rows of cavities and associated tooling (machine speed 2.0 rpm, die pressure 80 psi). Prior to encapsulation, fill material was mixed to form a uniform dispersion at ambient temperature. During encapsulation, fill material was held in a hopper at ambient temperature and under nitrogen blanket. Encapsulation was performed under yellow light.

Famotidine fill weight 150 mg; gelatin capsule shell weight 150 mg.

The ribbons obtained during encapsulation were smooth and the wedge temperature used 40.5° C. (105° F.) was adequate to achieve sealing of the capsules with good seams. No major issues were noted during encapsulation. Visually, the seams were satisfactory, and no leakage was observed during the encapsulation process.

Measured capsule weight was ranging from 240 mg to 260 mg with an average at 250 mg. Amount of famotidine per capsule was ranging from 19.4 mg to 19.7 mg.

Bulk famotidine softgels were bottled into 60 cc white HDPE bottles with CRC caps at a count of 90 softgels per bottle manually.

### 4.2—Famotidine Stability, in Softgel Capsule

Method: same assay as disclosed in paragraph “Chemical stability” above.

Bottled famotidine 20 mg softgels were placed in stability chambers at 40° C./75% RH, 30° C./65% RH and 25° C./60% RH, and monitored over time.

Famotidine assay stability data for up to 24 months of storage for Examples 6 and 7 batches of famotidine, 20 mg softgels are summarized in Table 10. Degradation summary data for the softgels capsules placed in stability chambers at 25° C./60% RH are presented in tables 11 and 12.

TABLE 10
Assay stability of bottled famotidine softgels, 20 mg during storage for 24 Months

| | Example 6 (inventive) | | | Example 7 (inventive) | | |
|---|---|---|---|---|---|---|
| | 25° C./60% | 30° C./65% | 40° C./75% | 25° C./60% | 30° C./65% | 40°/C. 75% |
| Months | RH | RH | RH | RH | RH | RH |
| 0 | 97.2% | 97.2% | 97.2% | 98.5% | 98.5% | 98.5% |
| | (19.4 mg) | (19.4 mg) | (19.4 mg) | (19.7 mg) | (19.7 mg) | (19.7 mg) |
| 1 | 96.6% | — | 95.6% | 97.7% | — | 97.0% |
| | (19.3 mg) | | (19.1 mg) | (19.5 mg) | | (19.4 mg) |
| 2 | — | — | 95.9% | — | — | 97.1% |
| | | | (19.2 mg) | | | (19.4 mg) |
| 3 | 96.2% | 95.9% | 94.7% | 97.2% | 97.6% | 96.0% |
| | (19.2 mg) | (19.2 mg) | (18.9 mg) | (19.4 mg) | (19.5 mg) | (19.2 mg) |
| 6 | 96.2% | 96.9% | 95.4% | 97.4% | 98.0% | 95.3% |
| | (19.2 mg) | (19.4 mg) | (19.1 mg) | (19.5 mg) | (19.6 mg) | (19.1 mg) |
| 9 | 95.6% | 96.5% | — | 96.5% | 96.7% | — |
| | (19.1 mg) | (19.3 mg) | | (19.3 mg) | (19.3 mg) | |
| 12 | 95.2% | 95.9% | — | 96.9% | 96.5% | — |
| | (19.0 mg) | (19.2 mg) | | (19.4 mg) | (19.3 mg) | |
| 18 | 94.8% | — | — | 96.4% | — | — |
| | (19.0 mg) | | | (19.3 mg) | | |
| 24 | 95.4% | — | — | 96.4% | — | — |
| | (19.1 mg) | | | (19.3 mg) | | |

TABLE 11
Famotidine degradation data for softgel capsule comprising
fill formulation of Example 6 stored at 25° C./60% RH.

| | Time Point (months) | | | | | | | |
|---|---|---|---|---|---|---|---|---|
| | Initial | 1 | 3 | 6 | 9 | 12 | 18 | 24 |
| Total | 0.585% | 0.634% | 0.657% | 0.775% | 0.950% | 0.952% | 1.194% | 1.165% |
| Impurities¹ (%) | | | | | | | | |
| FAM-A1² | 0.106% | 0.073% | 0.109% | 0.101% | 0.130% | 0.132% | 0.189% | 0.192% |
| FAM-A2² | NA | NA | 0.002% | 0.003% | 0.004% | 0.005% | 0.009% | 0.011% |
| FAM-A3² | 0.144% | 0.149% | 0.144% | 0.153% | 0.173% | 0.138% | 0.113% | 0.113% |
| FAM-A6² | 0.142% | 0.200% | 0.190% | 0.258% | 0.311% | 0.324% | 0.356% | 0.372% |

¹Total Impurities include all peaks that were detected.
²Known impurities of famotidine

TABLE 12
Famotidine degradation data for softgel capsule comprising
fill formulation of Example 7 stored at 25° C./60% RH.

| | Time Point (months) | | | | | | | |
|---|---|---|---|---|---|---|---|---|
| | Initial | 1 | 3 | 6 | 9 | 12 | 18 | 24 |
| Total | 1.057% | 1.080% | 1.147% | 1.245% | 1.635% | 1.468% | 1.739% | 1.675% |
| Impurities¹ (%) | | | | | | | | |
| FAM-A1² | 0.113% | 0.079% | 0.132% | 0.118% | 0.179% | 0.127% | 0.216% | 0.239% |
| FAM-A2² | NA | NA | NA | 0.003% | 0.004% | 0.005% | 0.011% | 0.013% |
| FAM-A3² | 0.169% | 0.150% | 0.152% | 0.164% | 0.178% | 0.172% | 0.130% | 0.110% |
| FAM-A6² | 0.527% | 0.584% | 0.550% | 0.565% | 0.766% | 0.637% | 0.573% | 0.578% |

¹Total Impurities include all peaks that were detected.
²Known impurities of famotidine

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. An oral dosage form comprising a soft gelatin capsule and a liquid fill contained within the soft gelatin capsule,
wherein the liquid fill comprises a suspension of an H2 receptor antagonist in a hydrophobic/lipophilic excipient substantially free from water, and
wherein at least 50%, in wt %, of the hydrophobic/lipophilic excipient consists of linoleic acid or linoleate moiety.

2. The oral dosage form according to claim 1, wherein the hydrophobic/lipophilic excipient has a HLB value below 3, preferably below 2, preferably at a maximum of 1.

3. The oral dosage form according to claim 1, wherein the hydrophobic/lipophilic excipient consists of glyceryl monolinoleate, corn oil, or combinations thereof.

4. The oral dosage form according to claim 1, wherein a solubility of the H2 receptor antagonist in the hydrophobic/lipophilic excipient is below 1 mg/g.

5. The oral dosage form according to claim 1, wherein the H2 receptor antagonist is selected from the group consisting of cimetidine, ranitidine, nizatidine, roxatidine and famotidine, and pharmaceutically acceptable salts thereof.

6. The oral dosage form according to claim 5, wherein the H2 receptor antagonist comprises famotidine.

7. The oral dosage form according to claim 1, wherein the H2 receptor antagonist comprises at least 15%, in wt %, of the liquid fill.

8. The oral dosage form according to claim 1, wherein the H2 receptor antagonist comprises at least 5 mg per oral dosage form preferably at least 10 mg.

9. The oral dosage form according to claim 1, wherein after 24 months at least 95% of the H2 receptor antagonist has not degraded.

10. The oral dosage form according to claim 1, comprising a soft gelatin capsule and a liquid fill contained within the soft gelatin capsule,
wherein the liquid fill consists of a suspension of famotidine in a hydrophobic/lipophilic excipient substantially free from water, and
wherein the hydrophobic/lipophilic excipient consists of glyceryl monolinoleate, corn oil or combinations thereof.

11. The oral dosage form according to claim 10, comprising a soft gelatin capsule and a liquid fill contained within the soft gelatin capsule,
wherein the liquid fill consists of a suspension of famotidine in a hydrophobic/lipophilic excipient substantially free from water, and
wherein the hydrophobic/lipophilic excipient consists of glyceryl monolinoleate.

12. The oral dosage form according to claim 10, comprising a soft gelatin capsule and a liquid fill contained within the soft gelatin capsule,
wherein the liquid fill consists of a suspension of famotidine in a hydrophobic/lipophilic excipient substantially free from water, and
wherein the hydrophobic/lipophilic excipient consists of corn oil.

13. A method of treating a gastric disease or disorder by use of the oral dosage form according to claim 1.
